(19) United States
(12) Patent Application Publication
(10) Pub. No.: US 2005/0236003 A1
(43) Pub. Date: Oct. 27, 2005

(54) Apnea nipple and oral airway and mandibular advancement device

(76) Inventor: Charles Robert Meader, Charlestown, NH (US)

Correspondence Address: CHARLES R. MEADER, 188 MAIN ST., BOX 347, CHARLESTOWN, NH, 03603, US

(21) Appl. No.: 11/023,085

(22) Filed: Dec. 28, 2004

Related U.S. Application Data
(60) Provisional application No. 60/562,523, filed on Apr. 16, 2004.

(51) Int. Cl. A61F 5/56

(52) U.S. Cl. 128/848

(57) ABSTRACT

The invention main, is a sleep apnea prevention device which is designed to move the lower jaw forward, keep teeth and lips apart, and guarantee full oxygenation needs with oral airway that is centered in an anterior dental-buccal space shield and wing portion. This, with mouth guard for lower teeth, is all a unit as a single piece of molded plastic or any other material; with said unit modeled from four theoretical portions including a shield like anterior portion fitted and anchored between anterior teeth-gums and behind the lips in the anterior buccal space with flanking wing like fins extending in that space laterally back to the upper second molars, thus allowing good retention in place whether mouth is open wide or minimally, or closed or moving side to side. Said shield is functionally tethered at the top front which becomes its fulcrum as it engages the lower teeth with a mouth guard portion and swings the lower jaw forward with bite activity; mouth guard pylon like blocks mounted on the mouth guard superior surface keep the teeth apart and help swing the jaw forward. Said shield in midline supports a nipple like projection which is, actually, a tube-like conduit which keeps the lips apart and becomes an oral airway. This device can be used alone or with CPAP face mask in place and user must coordinate with health provider to insure sleep apnea is only moderate and not just masked and inadequately treated. It usually does help snoring and bruxism.

### COMMENTS ON DRAWINGS AND FIGURES

FIG. 1 comment: DMAN device lateral view cut away showing nipple [N], Airway [A], upper and lower teeth [T], contour and bulk of the upper wing above [W] and the mouth guard below [M] with reinforcement posteriorly [R]. The entire device is held in place by tension of the lips trying to close in front and the buccal drag as well as bite force usually applied. Also it remains seated by being well fitted to the user. Thermo-plastic elastomer is used with harder plastic available as option.

FIG. 2 comment: DMAN saggital cut standing alone. Not shown here as the shield is vertical in this model but usually there is a tilt 10 degrees back from true vertical off the mouth guard. This allows the teeth to be opened unclenched without the entire shield tilting forward. It also alone keeps the lower jaw on tension forward as the shield is pulled to vertical by the lips. This view shows as above the wing shape at the midline and its contour [W], as well as nipple [N], shield [S] and mouth guard part [M]

FIG. 3 comment: DMAN from right above shows nipple [N], shield [S], wing [W] and pylons [P] on mouth guard portion [M]. Note the nipple aperture is oblate horizontal [NA] and that the flanking lateral air holes [AH]which can be two or four depending on the model allow increased oral air flow if needed as the lips are kept apart by the nipple outer shape. The teeth are kept apart by the upper shield at the top of the wing in the superior forchette of the anterior buccal space and below by the mouth guard portion on just the lower teeth. The pylons impacted by the upper molars on bite position keep the teeth apart even more and prevents contact and grinding of the teeth [bruxism] which is extremely common in sleep apnea.

FIG. 4 comment: DMAN device from left above with same indicator labels as FIG. 3 above. Note the tilt backwards of the shield from the vertical on the horizontal mouth guard portion. This 10 degree backward tilt brings the lower jaw forward as the bite is applied and counters the tilt tendency of the bulky upper wing. The offset is about 10 millimeters forward with tilt of the 2 and ½ inch shield from the top of the wing. The wing tips keep the device on the level stopping it from rocking forward.

FIG. 5 comment: DMAN device view from back above note the same indicator labels as FIG. 3. Note the airway holes to the side of the oblong nipple airway aperture. This adds supplemental oral air holes for those patients with total nasal passage occlusion. We have option for two shield holes on each side of the shield [see FIGS. 5 and 6]. The outer nipple contour keeps the lips apart allowing free flow orally through the lateral air holes. This also gives extra safety if mouth guard becomes shifted in the night. The tongue is kept away from the area by keeping the teeth/jaws widely separated by the pylons and also re-enforcing just behind the front guard portion.

FIG. 6 comment: DMAN device from the front with four flanking air holes option. Note nipple [N], shield [S], wing [W] and mouth guard portion [M]. Note the nipple aperture is oblate horizontal [NA] and that the flanking lateral air holes [AH] which can be two or four depending on the model].

FIG. 7 comment: DMAN Device which show saggital cuts of device with cross cuts of the wing shape With laminated like change in thickness [narrowing distally] widest at the center with extra bulk behind keeping the shield plane in front offset from the plane of the front teeth by 4 millimeters advancing the mandible as the lips and force on the pylons with bite make device vertical. Note also the underside of the wing allows sideways movement of air to smaller shield air holes keeping the tongue away from the channel.

FIG. 8 [old 12] comment: DMAN with full head views of user with device in place. Note there is enough material external to quickly pull out the device in a choking or vomiting emergency from other causes at resuscitation. Removal is easily accomplished by EMT, family, or user his or herself. Note the root of the nose is pulled forward by the bulk of the front top wing enough to open the nasal airway slightly in some users.

1. The anterior buccal shield part of the sleep apnea prevention device is the most important invention as it is such a novel design and shape with superior and lateral wings and three dimensional thickness of the top but not the lower part border providing the invented embodiments of this art as the upper wing becomes the pivot point to pull the lower jaw forward as it attaches with one mouth guard part to that lower jaw.

2. The wing [on the shield part in claim 1] in its thickness is well anchored with mouth either closed or open and still keeps the jaw [and tongue] forward; because of its bulk it moves the fulcrum point anteriorly and pushes the entire device and the lower jaw forward still more and is held tighter by the lips and cheeks.

3. It [the shield part in claim 1] supports the central nipple shaped airway [also unique]. Again in position with mouth open or closed; the bell like contour of the airway is a unique art with embodiment of keeping the airway secure in mid-position with better grip by labial tissue and fewer tendencies to slide sideways or in and out.

4. This nipple shaped airway with its unique art of the bell like outer contour prevents the airway falling back into the mouth and a longer exterior cylindrical extended airway is not needed, all because the nipple is held so well by the lips because of its shape. On cross section it is a flattened or oval shape as well, not round thus, this keeps it from twisting on itself in the long axis or rotating with patient movements.

5. Because the nipple protrudes just enough to grasp in emergencies, as in sudden vomiting or sneezing spell, it can be instantly removed in spite of its purposefully tight fit.

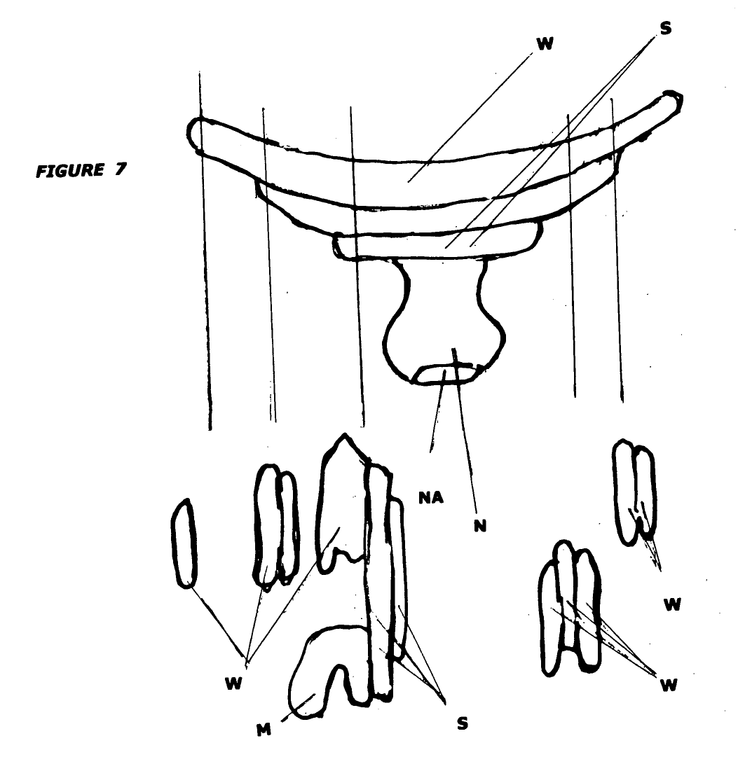

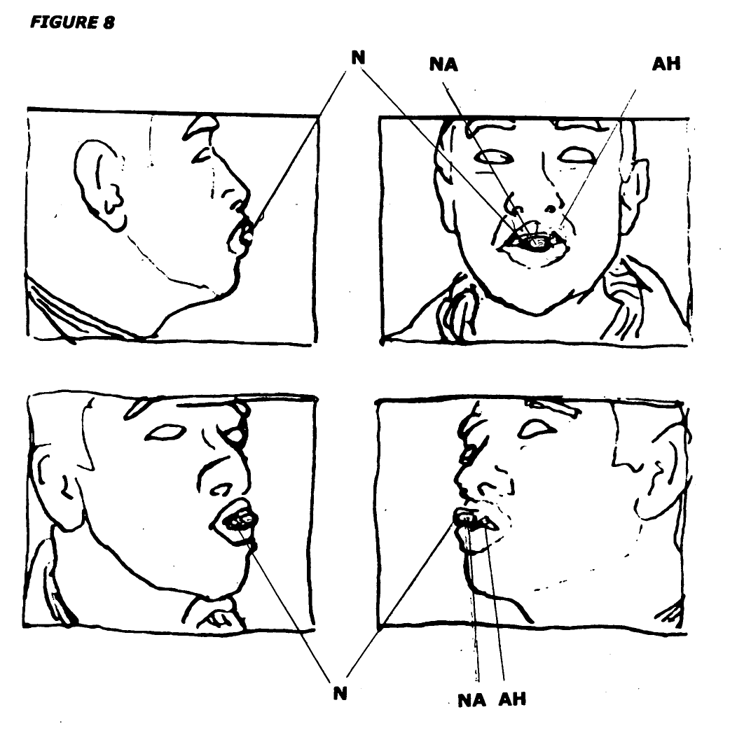

6. Is for the shape of the wing which on cross section has a channel just behind its seat on the shield which directs air to the central nipple channel or the two flanking holes in the shield. This keeps the tongue from blocking the shield with mouth open OR closed by allowing air to come from the side releasing any seal beginning. [See FIG. 7]

7. Is for shape of the wing part, thick in its main central portions tapering to a narrow edge at the top, narrow enough but not sharp so as to comfortably extend to the very fold between the upper lips/cheeks as it attaches to the gums; the more contact surface area the better it seats the device; better purchase also obtains with the rounded wing tips which are also thinner as the extend laterally; maximum contact is made, again for anchoring of the inter buccal-dental space but not so far back as to block Stenson's Duct which is the outlet of the parotid salivary gland opposite the second upper molars.

8. The nipple airway faces outward with bell like anterior widened shape and the surface of its own shield attachment itself swelling centrally like a mountain all work to keep the lips pushed forward and apart. Guaranteeing no lip seal occurs even with jaws in nearly closed position.

9. The nipple shaped central airway is supported by extra bulk of shield material on either side and the shape of the nipple sides all prevent the airway from collapsing or shifting laterally out of position and preventing lip closing even with too vigorous a bite of the device at pylon mouth guard portion.

10. The wider central airway in the nipple with the flanking air holes gives maximum oral airway patency and flow is continued and consistent with mouth open or near closed or even if device is in a slightly shifted position or attitude, so that nasal airway is NOT needed for adequate air exchange in a normal physiologic state. It can be used but is not needed.

11. The nipple central airway because it is supported entirely by the flange and shield-like anterior buccal-dental space with intimacy of contact, the jaws can open wide or close tight and the airway stay patent and in position.

12. Because the design angles the shield backward from the vertical above the nipple midplane about 10 degrees it has additional elastic recoil pushing the lower jaw forward and making a tighter ft in the buccal space anterior and to either side at the chin.

13. Because the pylons on the mouth guard portion superior surface allow clenched teeth and still continued airway function it gives a feeling of control to the user as the device is moved forward by the clenching; this is to titrate better air intake sensed by the user. Users are instructed to advance the mandible as far as it can forward and remain comfortable [8 mm is the optimum distance], the user sets the position before sleep by clenching but the once asleep even jaw dropping open does not matter with function.

14. There are pylon-like platforms on upper border surface of the lateral arms of the lower jaw mouth guard part that keep the teeth apart even with mouth closed so that anterior teeth do not interrupt the oral channel for airflow over the tongue, lower now in forward position.

15. Said pylon-like platforms [see claim 14 above] because of their thickness allow any boil and bite step to mold and fit from lower teeth in underside groove and allow better capture of the mandible to advance it but spares the softening at the upper teeth contact point as one only dips the lower portion of the device leaving the pylon platform upper surface smoother. In functioning position the entire device can and should slide forward along the upper teeth but not the lower teeth.

16. All this allows easy guaranteed mouth breathing in sleep apnea, and this prevents any vacuum build up on inspiration even with blocked nasal passages. There are no expected suction events on inspiration as the area is no longer a closed space. Usually with nasal blockage the collapsing inspiration force narrows the hypopharynx and pulls the tongue backward [retrusing force]; because no vacuum can build and the tongue is held forward collapse of the hypopharyngeal airway becomes less likely. Blocked nasal airway does not matter as it can no longer accelerate the closure of the lower airway. The oral route is sufficient with the DMAN device.

17. We have presented an oral appliance made of one piece molded plastic, which reduces, in SOME mild to moderate, obstructive sleep apnea events in adult patients. Dentist visits for fitting and customizing is not required. It is safe to use as there are no buttons, screws, or adjustable hardware that can pop off and be inhaled as choking foreign bodies in the larynx. The cost of manufacture is minimal. This will be offered in hard or soft plastic and in three sizes with user sending us a bite imprint, note of height, age, weight, and sex. Since there will be some for whom it is ineffective or not well fitted, a money-back guarantee up to 90 days is reasonable.

18. With a therapy plan of changing as many parameters of the sleep apnea pathophysiology as possible, more chance for success is provided by the new art HEAD STRAP [see FIG. 10] and discussed below as a dependent claim. Provider instruction for lifestyle changes and better sleep hygiene is given but often wedge pillow, bed block, for GERD or even soft cervical collar keeping the chin up [and out] will make a difference in success rate.

19. Finally for all the previous arts for anti-snoring devices for treatment, that are similar here in any way, in that long list of antecedent U.S. Patents listed in this document below, if there is overlap of claims, we claim a new obstructive sleep apnea treatment embodiment [use], with that overlapped claim. Anti-snoring is NOT what we are about although it is reduced with prevented sleep apnea. We increase airway competition with the nasal passage and can actually worsen some elements of snoring noise production, such as vibration of the palate in high speed air movement, which may occur de novo or worsen previous case.

20. Is for primary but not exclusive use for obstructive sleep apnea, on the other hand not just for anti-snoring. There is real benefit when the “pause gasp snort” of recovery seen in sleep apnea is stopped. Snoring is much improved as a secondary benefit. There is no guarantee that this will for apnea in any given case. We have developed a low cost apnea sound [acoustic] analyzer electronic device [see second dependent claim below] which compares before and after recordings to prove treatment effectiveness. Inevitably a health professional must decide. All customers are expected to coordinate with their own doctors with implementation of the DMAN.

21. The very bulkiness of the entire device from the thickened midline wing structure, but also the shield and mouth guard portions that insert inside the upper lip in front of the teeth pushes the root of the nose forward and opens the nostrils allowing some people a better nasal airway.

22. The pressure of the clenching of the teeth against the pylons pushing them down to firmer seat in the back molars tends to push the jaw forward as it swivels on fulcrum at the upper shield against the upper front teeth anterior surface in part because there is such a deep recess above of the gums to the upper lip connection at the frenulum and because of the deep reinforced notch below anteriorly in the mouth guard well seating the lower jaw anterior teeth.

23. With the design, because upper surface of the one mouth guard potion is smoother and with pylon is not so easily indented with upper teeth on any boil and bite step or over time, it will slide along the upper teeth forward and back, unlike the bottom teeth which are well captured, thus the entire guard is allowed to come forward carrying the jaw and tongue with it.

24. With the unique design the guard stays engages or seated in all jaw positions without losing purchase in the upper anterior buccal space or the mouth guard element below on the mandible below, and this keeps the jaw from wobbling or moving from side to side, and this helps prevent jaw joint problems such as TemporoMandibular Joint syndrome [TMJ].

25. Next, is the under-bite reinforcement feature which guarantees the tight connection of the lower incisors into the underside of the apnea device; this is reinforced by a large but level block of plastic set-lower and behind the anterior dental groove making a deeper front trench that receives the lower jaw very front teeth, the incisors; this prevents slippage fore and aft, thus guaranteeing the movement and retention of the jaw forward.

26. Because the pylon on top surface above and the mouth guard portion below allow teeth clenching that is safe, neither grinding teeth nor hurting the jaw joint [the teeth are 17 mm or more apart at function bite with force], the so called bruxism reinforcement response can open the nasal passages; if one bites hard, clenches teeth, suddenly the lower part of the turbinates allow more nasal air movement. This may be mechanical or reflex but is real and most people surveyed have this reflex, but not all. This helps nasal air movement.

27. There is a lateral flare of the lower outer border of the mouth guard portion of the device on either side extending from back end to the beginning of the front curve; this is a unique embodiment of a new art and that is: it is shaped especially to engage the cheek buccal tissue surface and with that inward friction/force keep it from slipping backward. Vector of force central on the shape pushes it forward like pip of an orange or a whale shape. This is another example of the manifold and subtle forces pulling, pushing, and swinging the lower jaw forward with the original DMAN design.

28. The apnea prevention device by its nature prevents bruxism or teeth grinding because it keeps the teeth from actually touching upper and lower teeth surfaces at all because of the interposed mouth guard portion facing down. It also reduces most of the noise of snoring, so it is sometimes an effective anti-snoring device, but this is not its purpose. It is primarily a guarantee of oral airway statues and mandibular advance. With a good patent nasal passage it may increase the snoring with new though not necessarily louder snoring sounds as the maximum air flow shifts back and forth from nasal path to oral pathway thus vibrating the uvula and soft palate in a new way. See the last claim b43 [DMAN use with nipple plugged up].

29. Variations on the device are several: three sizes; small, medium and large; two angles of shield back tilt on mouth guard portion; 10 degrees or 15 degrees for 8 mm advance and 12 mm advance range of mandibular advancement splint effect, Two plastic options: soft thermoplastic and hard acrylic; this emphasizes that the boil and bite step is NOT needed but optional with the DMAN device and also that no dentist visit is needed for adjustment as sizes and style are selected before the sale with charts and nomograms including age, sex, height, bite imprint, bite imprint with jaw forward, and max width of central dentate line sedation accomplished actively by user.

30. The wing and upper jaw work in concert to bring the lower jaw forward and the user has an active role in placing all this lower mouth guard part forward, at least until sleep begins. This is marked by the nipple pointing up as the lower end of the flange comes forward and tilts backward at the top. The nipple pointing up and not down when in position, means the chin is forward. We call the nipple up position the gold standard end point. It allows the user some feedback on his efforts to jut forward the mandible, as he readies for sleep.

31. Lateral upper wing upper border abuts the inverted gutter of superior forchette between the gums and cheek. This is the lock which stops the nipple pointing down as the upper border will be forced superior to rotate the device. If the entire device rotates forward it ruins airway function by letting the mandible drift back. The upper gum line lateral and posterior prevents this, since the border of the upper lateral wings impinge at the high point and cause pain if forced. This is made certain by making the wingspan as long as possible [120 mm or maximum] for that user; he or she could cut off the distal 15 mm if it is too long.

32. The entire distal wing is now flat in cross section so there is adequate support top-bottom even if 120 mm wingspan. To increase the drag and contact zone of the important wing tips with cheek [so as to prevent it always going to the full stop on rotational force into the forchette position] there is a widened end to the wingtip. This is the so called spoonbill tip: a large coin-like shape in silhouette but flat; this makes an unusual and distinct shape of the wing tip as it tapers to the tip [span of 120 mm]. (Not shown)

33. The previous mentioned and utilized flare projection at lateral lower back mouth guard may in fact compete with forward or up movement at front by reducing skin slack and limiting the upward movement of tissue cheek. It should be kept in place but narrowed as it still supports the relatively tall pylon to it as inside and does increase drag at the cheek even with thin cross section.

34. Front lower teeth are the key to moving the mandible forward and keeping it forward in nipple-up station. They require a good fit or seat into the plastic mouth guard; if this mouth guard is allowed to slide back and forth there is no user confidence built. There is also less tension possible for bringing that jaw forward and keeping the nipple pointed up in gold-standard position. It is clear that the user becomes active as the jaw seeks optimum position just before sleep. If the user bites then drifts the jaw forward, there is a feeling the airway is maximized and this is instinctual but also taught in instructions for use as well. For better control it must not slide variously forward or back in its mouth guard slot but be tightly seated but most important strongly blocked from drifting back.

35. A block of plastic reinforcement is placed behind and inside of the front lower teeth; this works to keep the front teeth engaged. We use cheap mouth guards thin 2 mm and fill in the gutter against the inside wall and we add substance to inside or tongue side of that line. This gives an asymmetric cross section with 4 times the thickness to the posterior internal mouth guard wall as the anterior buccal side. This bridges the arc of the inside curve of the anterior mouth guard: the chord of that arc is a bridge under the tongue which anchors the entire tongue side of the mouth guard giving much strength. Behind the lower incisors or front central teeth and spreading back to lateral front teeth this bridge gives strength to the structure and firmness to the mouth guard now locked in place. This claim is the under-bite reinforcement feature, which guarantees the tight connection of the lower incisors into the underside of the DMAN device; it is a reinforced large but level block of plastic set lower and behind the groove making a deeper front trench that receives the lower jaw very front teeth, the incisors; this prevents slippage fore and aft, thus guaranteeing the movement of the jaw forward. This patent claim will be called the “under-bite reinforcement feature.”

36. This lower bridge also fills in much of the space under tongue. The advantage to that is that it brings the tongue up and tends to pull it forward by keeping it off the floor of the mouth, especially as the mandible falls open it will tend to bring the tongue forward.

37. Pylons have to be anterior and not so tall in back else user will complain of TMJ symptoms. 12 mm+5 mm off the teeth surface is too much at the extreme rearward back teeth but may be ok extreme anterior which is 30 mm forward. In addition to the 5 mm from the big mouth guard the most height is 4 mm at third molar, 8 mm at first molar, and 12 at premolar or bicuspid. Along the curve of the lower jaw the lateral straight way begins just off the corner moving back 25 to 30 mm to end of teeth line. [Back at 5+4, mid position 5+8, and forward at the curve, but not interfering with the nipple wing structure, the 5+12 mm might be the max].

38. Options for two piece construction and the matched connections fitted together at home by the user afford potential for a more custom fit. Upper part sizes may be different than lower part sizes with possible disposal of either part. Wing structure separates from base with wing fused to nipple and flange. We will start that way. If these separate halves are of equal bulk, there will be no ‘parts’ to fall or break off or choke the user in the night. So safety issues are covered.

39. This separate part works best with boil and bite methods as only the mouth guard part does the hot boiling water step; this is fine as long as dummy connector is in place while boiling so as not to collapse the female part of the connecting slot or tongue in groove; it can also connect in three different areas with sliding snap like connection.

40. Nipple up attitude is the best position for jaw forward function. This is expected because the nipple flange is connected in a firm but elastic way with the full wing above it and in a NON-elastic way [seat slot/connector snap] to the mouth guard part below the lower flange can slope back more and have thicker strut like vertical borders on either side to give it stiffness and let it tilt back 5 degrees [like an old fighter plane windshield] even when not engaged. Force forward is transmitted from above by the full combined nipple flange stiff support pillars lateral [not all but SOME hinge elastic effect.] It must tilt back: the angle of the nipple mount or flange must be NOT 90 degrees incident the mouth guard portion but leaning back slightly [say 5-10 degrees again with the nipple pointing up slightly.

41. Option for mouth guard shape: there is a flare of the lower outer part of the mouth guard portion of the device on either side extending from back end to the beginning of front curve; this is a unique embodiment of a new art and that is: it is shaped specially to engage the cheek buccal tissue surface and with that friction on contact keep it from slipping backward. This is another example of the manifold forces pulling and pushing the lower jaw forward (much like a scapula floating in the muscle of the upper limb girdle but not attached at all except by the collar bone at the sternum in front.)

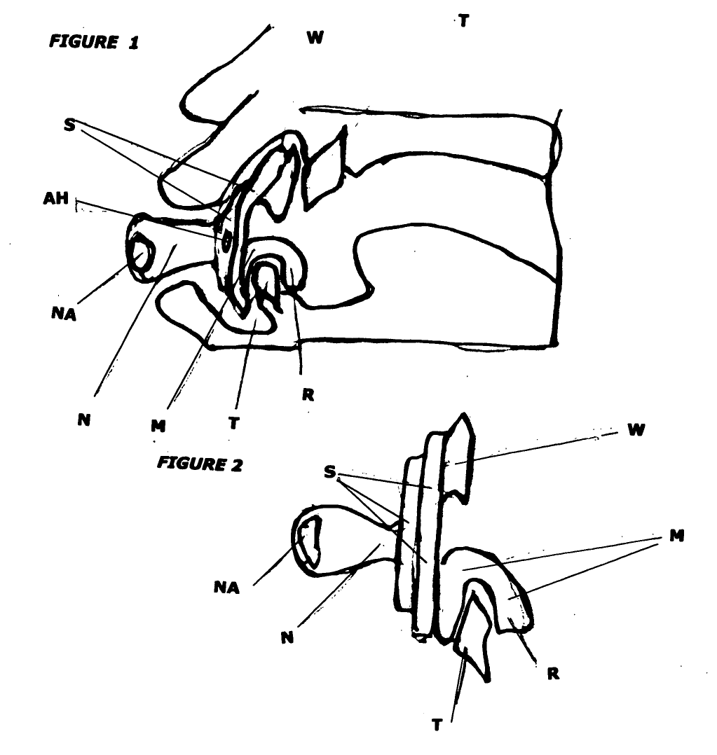

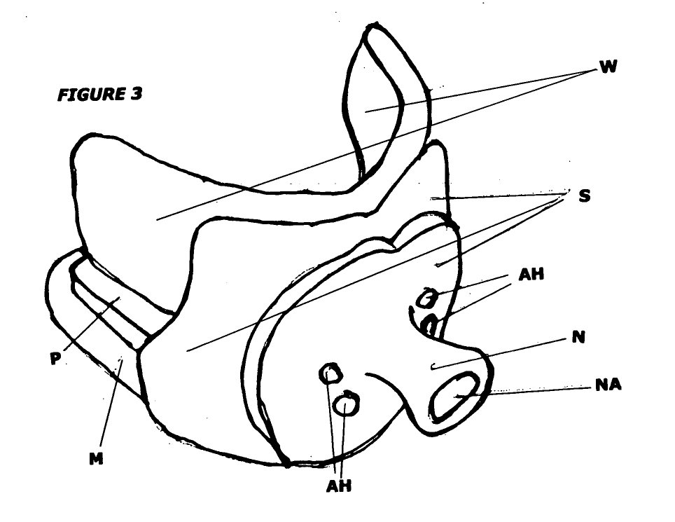

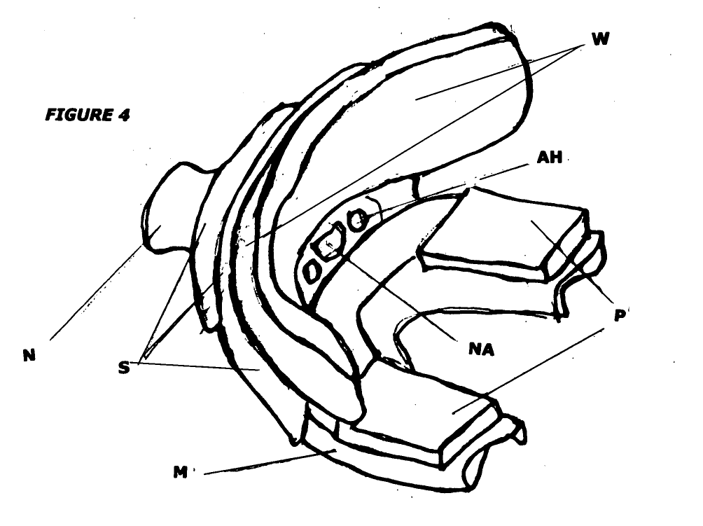

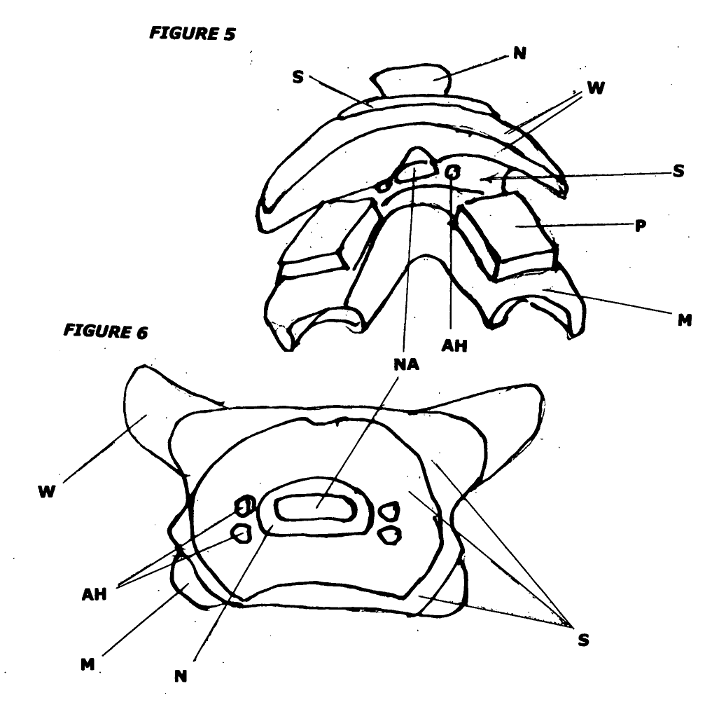

42. The vectors of forces pushing and pulling and swinging the mandible forward are unique in this art. There are three. They do not depend on rigid connection of the top and bottom dental bite line but rather the seating of a tight and bulky fit of the nipple shield in the anterior buccal space in front of the top front teeth and the taught lips in front. The first is the wider set-back of the bulk of the upper wing which keeps the top of the apnea nipple out from the vertical plane of the top front teeth. The tendency to rock forward is countered by the lips which hold the shield vertical in that natural position, pulling the mandible forward. Second is the 10 degree tilt backward of the vertical nipple flange-shield on the mouth guard portion base held rigid by the extra bulk of the lateral struts on either front side of the flange-shield and with any bite force the upper front teeth prevent the tilt and so the mandible drifts forward. Third is the pendulum like motion of the entire device with upper front teeth and gums the fulcrum [see FIG. 1 . . . ]; as the top molars impact on the pylons seated on the mouth guard portion the force is directed down but because the device is held tightly against the top front teeth there occurs a fulcrum with resultant vector of force swinging the mandible forward. Indeed the user can feel this and intuitively improves the airway as he or she varies the force of the bite while seeking optimum positioning. As a result of the shape and design of the DMAN the mandible is advanced improving pharyngeal airway, snoring, and apnea. Teeth grinding is impossible because upper and lower teeth do not touch. Mouth breathing becomes effective with a patent nipple portion.

43. We teach that this art, the DMAN Mandibular advancement and oral airway appliance, can be used another way. This major embodiment is its use entirely WITHOUT the oral airway. It has been used successfully without the oral airway or air-holes, with and without CPAP, as it still keeps the mandible advanced and allows the nasal passage to provide all the air movement if it can. This baffled, blocked or dummy version is pictured in FIG. 23 at the bottom [with the Coin-wing and Bulged Mouth guard version as alternatives embodiments or models]. Note the nipple shape is intact so as to keep the device positioned well but the central airway is eliminated. By making all the air movement through the nose, optimum moisturizing occurs and the flutter of the soft palate as a snoring noise source is muted. Bruxism is treated, snoring is helped and sleep apnea episode frequency is usually decreased.
